(19) United States
(12) Patent Application Publication
(10) Pub. No.: US 2023/0323382 A1
(43) Pub. Date: Oct. 12, 2023

(54) B3 TRANSCRIPTION FACTOR GENE FOR SIMULTANEOUSLY IMPROVING LENGTH, STRENGTH AND ELONGATION OF COTTON FIBERS AND USE THEREOF

(71) Applicant: ZHEJIANG UNIVERSITY, Hangzhou (CN)

(72) Inventors: Tianzhen ZHANG, Hangzhou (CN); Zegang HAN, Hangzhou (CN); Yiwen CAO, Hangzhou (CN); Yan HU, Hangzhou (CN); Lei FANG, Hangzhou (CN)

(21) Appl. No.: 18/313,288

(22) Filed: May 5, 2023

Related U.S. Application Data
(63) Continuation of application No. PCT/CN2021/118917, filed on Sep. 17, 2021.

(30) Foreign Application Priority Data
Mar. 23, 2021 (CN) 202110309020.7

(51) Int. Cl. C12N 15/82; C12Q 1/6895; A01H 6/60

(52) U.S. Cl. CPC C12N 15/8262; C12Q 1/6895; A01H 6/604; C12Q 2600/13

(57) ABSTRACT

Providing a B3 transcription factor gene for simultaneously improving length, strength and elongation of cotton fibers. The cDNA sequence of gene GHFLS in tetraploid upland cotton TM-1 is SEQ ID NO. 1, and the genome sequence is SEQ ID NO. 2; GHFLS contains a non-synonymous mutation SNP, located at 1391 bp of the coding region with the base changing from A to G and the corresponding amino acid changing from Lys to Arg. The GHFLS gene was overexpressed in Arabidopsis thaliana caused a significant reduction in the root length of the T2 generation, demonstrating its important role in the cell elongation mechanism. The fiber quality of the cotton variety (line) with haplotype AA is significantly better than that with haplotype GG. The gene has important research value and application prospect in efficiently identifying high-quality fiber upland cotton varieties, improving cotton fiber quality and cultivating new varieties of high-quality cotton fibers.

### CROSS-REFERENCE TO RELATED APPLICATIONS

The present application is a National Stage of International Application No. PCT/CN2021/118917, filed on Sep. 17, 2021, which claims priority to Chinese Patent Application No. 202110309020.7, filed on Mar. 23, 2021, both of which are hereby incorporated by reference in their entireties.

### REFERENCE TO SEQUENCE LISTING

The present application is being filed along with a Sequence Listing in electronic format. The Sequence Listing is provided as a file entitled DF225166US-SEQUENCE LISTING ST.26, created on Mar. 23, 2023, which is approximately 14.1 Kb in size. The information in the electronic format of the Sequence Listing is incorporated herein by reference in its entirety.

### TECHNICAL FIELD

The present application belongs to the application field of biotechnology and relates to a B3 family transcription factor gene related to length, fiber strength and fiber elongation of cotton fibers.

### BACKGROUND

As the main source of natural fiber, cotton is an important cash crop. Cotton production not only has an important influence on the development of China's agriculture and even the national economy, but also plays an important role in the world cotton trade market. In addition, cotton fiber is an excellent and most widely used natural fiber, and it is also an important raw material of textile industry, which plays an important role in the development of national economy. With the improvement of people's living standard, the demand for natural pure cotton fabrics is increasing, and the requirements for fiber quality are getting higher and higher. Therefore, it is particularly important to dig deeply and utilize genetic variation related to cotton quality.

Genome-wide association study (GWAS) is a new strategy, which takes millions of single nucleotide ploymorphism (SNP) in the genome as molecular genetic markers, carries out the correlation analysis at the genome-wide level, and finds out the gene variations that affect complex traits through comparison. With the improvement of genome sequencing technology and the reduction of sequencing cost, combined with the high development of bioinformatics, GWAS has become one of the most effective methods to dig and analyze genes of human diseases, crop agronomic traits and resistance traits and their related genetic mechanisms. By using genome-wide association study to mine and clone genes related to agronomic traits, GWAS has strong detection ability and high precision without the need of presupposing candidate genes, and thus is a hot spot in molecular breeding research. Belo et al. (2008) analyzed 8,950 SNPs of 553 excellent inbred lines by GWAS, and identified the loci related to oleic acid content, which was the first true genome-wide association study of maize. Huang et al. (2011) re-sequenced 517 rice landraces with the second-generation sequencing technology and obtained millions of SNPs. Then, 14 agronomic traits of rice were analyzed by GWAS, and 80 loci associated with traits were successfully identified. In addition, they re-sequenced as many as 950 rice populations, analyzed the flowering period and 10 yield-related traits by GWAS, and identified many known functional genes (Huang et al. 2012). Lin et al. (2014) re-sequenced the genome-wide of 360 tomato germplasm from all over the world. Through population differentiation analysis, it was found for the first time that the key mutation locus that determines the color of pink fruit peel, that is, the 603 bp deletion of the promoter region of SIMYB12 gene, inhibited the expression of this gene, thus making the mature pink fruit tomato peel unable to accumulate flavonoids, resulting in the difference between fresh and processed tomatoes. Zhou et al. (2015) re-sequenced 302 wild, local and improved soybean varieties, and found by GWAS analysis technology that 96 GWAS-related loci were related to previously reported QTLs, and identified new related loci related to oil content, plant height and fuzz production. Fang et al. (2017) identified 25 selection signals in the process of cotton improvement through genome-wide re-sequencing of 318 upland cotton materials. Through GWAS analysis, a total of 119 associated loci were identified, of which 71 were related to yield, 45 were related to fiber quality, and 3 were related to verticillium wilt resistance (Fang et al, 2017). Ma et al. (2018) re-sequenced and analyzed 419 core germplasm upland cotton materials, and found that 7383 SNPs were significantly related to these traits, located in or near 4820 genes. Some candidate genes that control flowering, affect fiber length and fiber strength were analyzed emphatically (Ma et al., 2018). Liu et al. (2021) used 290 natural populations of upland cotton cultivars to conduct genome-wide association study on cotton wilt resistance after years of field identification by combining with high-density SNP markers, and identified the main resistance locus Fov7, and determined that the gene GhGLR4.8 is a new plant atypical main resistance gene (Liu et al., 2021). The above results fully show that genome-wide association study has high positioning accuracy, even reaching the level of single gene. Using the obtained functional markers related to the target traits to screen the target traits can greatly speed up the breeding process and efficiency.

There are many kinds of plant transcription factors, which are involved in various signal transduction pathways and the process of growth and development. They are the largest functional category in eukaryotes, accounting for about 8% of the genome-wide (Weirauch and Hughes, 2011). Common plant transcription factors are MYB, AP2/EREBP, NAC, bZIP, homeobox, zinc finger, MADS, WRKY, B3, YABBY, Dof, etc. In addition, B3 family is a transcription factor family unique to plants and widely existing. B3 family contains B3-DNA binding domain, which plays an important regulatory role in plant growth and development by binding specific DNA sequences. According to the structural characteristics and functions, B3 family can be divided into five subfamilies: ARF family, ABI3 family, HSI family, RAV and REM subfamilies. These gene families play an important role in regulating plant growth and development, organ morphogenesis, flower bud differentiation and responding to various stress (Liu Yinghui et al., 2017).

### SUMMARY

The present application aims to provide a B3 transcription factor family gene Fiber length and strength related (GHFLS). Genome-wide association analysis shows that the gene is closely related to cotton fiber length, fiber strength and fiber elongation, which are three important fiber quality traits.

Another object of the present application is to provide use of the gene.

The object of the present application can be achieved through the following technical solutions:

A B3 transcription factor family gene GHFLS, where the cDNA sequence of the B3 transcription factor gene GHFLS in tetraploid upland cotton TM-1 is SEQ ID NO. 1, and the genome sequence is SEQ ID NO. 2; the transcription factor gene GHFLS contains a non-synonymous mutation SNP locus located at 1391 bp of the genome sequence; a base of this SNP locus mutates from A to G, and the corresponding amino acid changes from Lys to Arg; in addition, fiber length, fiber strength, fiber elongation and other fiber quality traits of cotton varieties with genotype AA are significantly higher than those of cotton varieties with genotype GG. Interestingly, many varieties bred in Xinjiang have a haplotype of GG, which is of great use value.

Use of the transcription factor gene GHFLS of the present application in identifying an upland cotton variety with high quality fibers.

Use of the transcription factor gene GHFLS in improving cotton fiber quality traits.

Use of the transcription factor gene GHFLS of the present application in cultivating a new variety with high-quality cotton fibers by genetic engineering.

A primer pair for detecting the SNP locus, where the upstream primer is SEQ ID No. 3, and the downstream primer is SEQ ID No. 4.

Use of the primer pair in screening high-yield cotton varieties.

A method for screening a high-yield cotton variety, including detecting one SNP locus, and selecting cotton with the base at 1391 bp of the genome sequence being A as a cotton variety with high-quality fibers.

The present application has the following advantages:

The present application excavates a B3 transcription factor family gene GHFLS closely related to cotton quality traits, fiber length, fiber strength and fiber elongation, which are three important fiber quality traits at the same time, by weight sequencing and genome-wide association analysis of cotton varieties. The transcription factor gene GHFLS of the present application is closely related to cotton quality traits in the genome-wide association analysis. The GHFLS cDNA and genome sequence provided by the present application are obtained by PCR technology, which has the advantages of small amount of starting templates, simple and easy test steps and high sensitivity.

The expression levels of GHFLS in different tissues and development stages of cotton were analyzed by transcriptome sequencing. The gene was preferentially expressed in ovules of cotton 3 and 1 days before flowering, and ovule seeds of cotton 1, 3, 5, 10 and 20 days after flowering, which indicated that the gene was related to fiber quality traits.

The SNP genotype of GHFLS in relatively high fiber quality and low fiber quality varieties was verified by PCR, which is easy to operate, sensitive and accurate.

Over-expression of the GHFLS gene in the model plan, Arabidopsis thaliana showed that over-expression of the GHFLS gene significantly shortened the root length of T2 generation Arabidopsis thaliana, which proved the important role of the GHFLS gene in cell elongation mechanism.

According to different SNP genotypes of GHFLS, the varieties can be divided into two groups. Statistical analysis shows that there are significant differences in fiber length, fiber strength and fiber elongation between the two groups, which further proves the correlation between this gene and cotton quality traits.

### BRIEF DESCRIPTION OF DRAWINGS

FIG. 1 shows the GWAS correlation analysis results of different yield traits of cotton;

FE, FS and FL represent fiber elongation, fiber strength and fiber length, respectively; the abscissa indicates the position (Mb) on the chromosome, and the ordinate indicates the significance of SNP locus association, which is represented by −log₁₀(P value).

FIG. 2 shows the expression levels of GHFLS in different tissues and development stages of cotton;

the abscissa represents different tissues, including Root, Stem, Leaf, ovule and fiber; the ovule tissue includes those collected 3 and 1 days before flowering, the day of flowering and 1 to 25 days after flowering, and the fiber tissue includes those collected 5 to 25 days after flowering.

FIG. 3 shows the sequence information of GHFLS and identification of different haplotypes;

there is a non-synonymous mutation SNP locus in the GHFLS sequence in the variety population, which is located at the position of 1391 bp in the genome sequence; the base of this SNP locus changes from A to G, and the corresponding amino acid changes from Lys to Arg.

FIG. 4 shows the comparative analysis of yield traits among different haplotypes of GHFLS;

the box represents the distribution of quality traits of the variety population; the abscissa refers to different planting environments, and the ordinate refers to the corresponding quality traits, namely fiber elongation, fiber strength and fiber length; there are 280 and 118 varieties containing AA and GG haplotypes respectively; white represents the distribution of quality traits of haplotype AA, and black represents the distribution of quality traits of GG; the horizontal line in the box represents the median value of character distribution; * * means there is a difference at the level of 0.01; * means there is a difference at the level of 0.05.

FIG. 5 shows that the GHFLS gene negatively regulates the root development of Arabidopsis thaliana;

the left box diagram represents the root length statistics of transgenic Arabidopsis thaliana and wild type; the ordinate is the root length of Arabidopsis thaliana, and * * indicates the difference at the level of 0.01; the photo on the right is the root growth photo of transgenic Arabidopsis thaliana and wild type in different strains.

### DESCRIPTION OF EMBODIMENTS

### Example 1 Excavation of Transcription Factor Gene GHFLS Related to Cotton Quality Traits

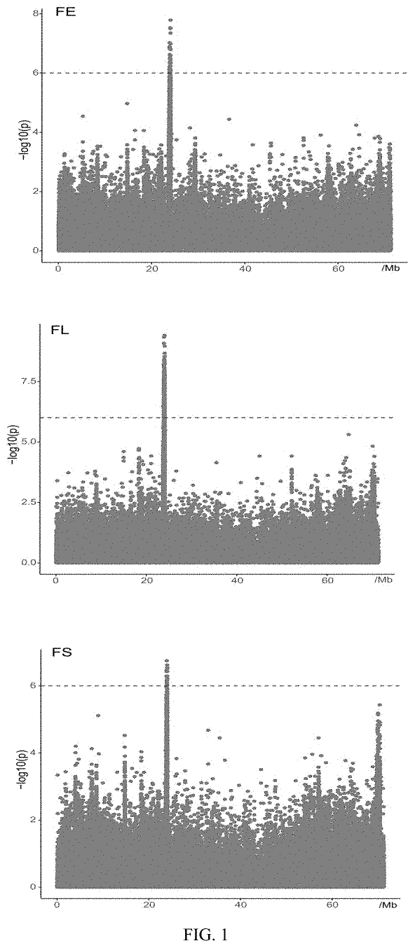

According to 486 modern upland cotton varieties or strains, the quality traits (fiber elongation, fiber strength, fiber length, micronaire value, fiber uniformity) were investigated in detail from 2016 to 2017 by planting three replicates in each variety field in Korla, Xinjiang and Shihezi, Xinjiang. At the same time, the these 486 cotton varieties (lines) were subjected to genome-wide re-sequencing, and 7.55 Tb sequencing data were obtained, with an average sequencing depth of 10.51×. These sequences were compared to the genome sequence of cotton upland cotton TM-1, and the whole genome SNP was identified by bioinformatics software. A total of 4 489 601 high-quality SNPs (a minimum gene frequency >0.05) were excavated for subsequent analysis. Firstly, genome-wide association study was performed, and then SNP signal correlation loci were screened according to P<1×10⁻⁶. By analyzing these correlation loci, we found that a SNP signal correlation locus (D11:23877270) on D11 chromosome can simultaneously correlate three quality traits of fiber elongation, fiber strength and fiber length (FIG. 1). This SNP locus was located in the exon region of the gene, and causes the mutation of the amino acid sequence. The gene is a B3 transcription factor family gene named GHFLS.

### Example 2 Acquisition of Transcription Factor Gene GHFLS

A cDNA sequence and a genome sequence of GHFLS were obtained from the genome sequence of upland cotton, see SEQ ID NO. 1 and SEQ ID NO. 2. According to the two ends of cDNA, full-length primers were designed for PCR amplification, and the primer sequences were F1: SEQ ID NO. 3 and R1:SEQ ID NO. 4. The PCR reaction procedure was as follows: pre-denaturing at 94° C. for 5 min; denaturing at 94° C. for 30 sec, annealing at 60° C. for 1 min, stretching at 72° C. for 1 min for 30 cycles; at last, extending at 72° C. for 10 min. The PCR products were sequenced and compared with cDNA to determine the accuracy of the sequence.

### Example 3 Expression Level Analysis of GHFLS in Different Tissues and Development Stages of Cotton

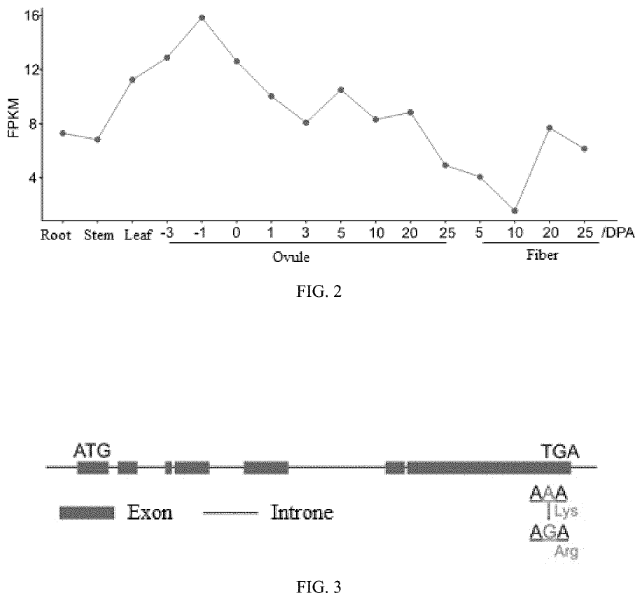

In this experiment, RNA samples from different tissues and development stages of cotton TM-1 were collected for transcriptome sequencing. The samples included roots, stems, leaves, ovules and fibers. Ovule tissue included those collected 3 and 1 days before flowering, the day of flowering and 1 to 25 days after flowering. The fiber tissue included the those collected 5 to 25 days after flowering. Transcriptome sequencing was carried out on an Illumina HiSeq 2500 platform, and the average sequencing depth of each sample reached 6 Gb. The gene expression level was calculated by comparing the sequenced reads with the upland cotton genome, and the calculated expression level was expressed by the number of sequencing fragments contained in every thousand transcription sequencing bases per million sequencing bases (FPKM). The experimental results are shown in FIG. 2. This gene was preferentially expressed in ovules of TM-1 cotton 3 and 1 days before flowering, and ovules and seeds of 1, 3, 5, 10 and 20 days after flowering, which indicated that this gene was related to fiber quality traits.

### Example 4 Use of B3 Transcription Factor Gene GHFLS in Identifying High-Quality Cotton Varieties and Improving Quality Traits

Based on the position of SNP locus (D11:23877270) on chromosome D11, genome amplification primers were designed at both ends, and the primer sequences were F2: SEQ ID NO. 5 and R2: SEQ ID NO. 6. By using this pair of primers, the DNA of 486 varieties was amplified by PCR and sequenced. The PCR reaction procedure was as follows: pre-denaturing at 94° C. for 5 min; denaturing at 94° C. for 30 sec, annealing at 58° C. for 1 min, and stretching at 72° C. for 45 sec for 30 cycles; at last, extending at 72° C. for 10 min. According to the sequencing results, the genotype of each population at the SNP locus was analyzed. It was confirmed that the GHFLS sequence contained a non-synonymous mutation SNP locus, which was located at the position of 1391 bp in the genome sequence. The base of this SNP locus changed from A to G, and the corresponding amino acid changed from Lys to Arg. According to the base information of this SNP locus, modern upland cotton varieties (lines) can be divided into AA and GG haplotypes (FIG. 3).

According to that genotype of SNP pair at the 1391 bp position of the GHFLS genome sequence, 280 haplotype AA material and 118 haplotype GG materials were identified from this natural population (FIG. 3 and table 1). Among them, Deltapine cotton 15, Stoneville cotton 2B, 108ϕ, KK1543, 611 [symbol] , which have made outstanding contributions to the breeding of upland cotton varieties in China, are all high fiber quality haplotypes AA, which further reflects the far-reaching influence of cotton varieties in America and Central Asian countries such as and Uzbekistan on the improvement of cotton varieties in China (Fang et al., 2017; Han et al., 2020). Most varieties bred in Xinjiang are GG haplotype, which indicates that high fiber quality haplotype AA has important utilization value.

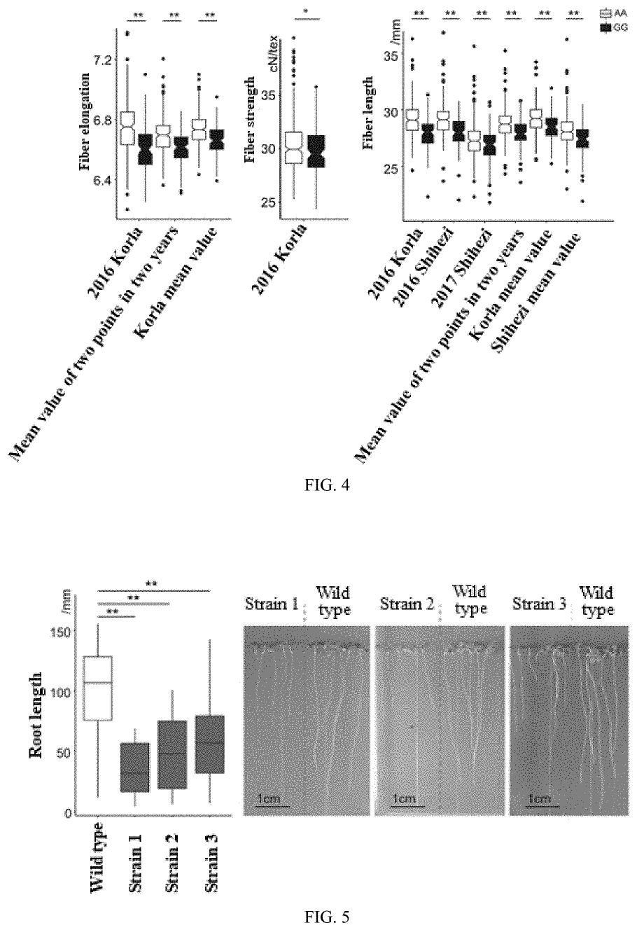

By using t-test statistical test method, the correlation of quality traits between two groups of haplotypes was calculated (FIG. 4). The results showed that the fiber elongation of haplotype AA was superior to that of GG haplotype cotton in three environments, namely, the average values of Korla in 2016, two points in two years (2016 Shihezi, 2016 Korla, 2017 Shihezi, 2017 Korla) and the average values of Korla in 2016 and 2017, showing extremely significant differences (P<0.01); the fiber strength of haplotype AA was also significantly higher than that of GG haplotype material in Korla environment in 2016 (P=0.032); at the same time, the fiber length of haplotype AA was superior to that of GG haplotype materials in the average of two points in two years in 2016 Korla, 2016 Shihezi, 2017 Shihezi, and the averages of Korla in 2016 and 2017 and the averages in two years in Shihezi (P<0.01).

A GHFLS gene overexpression vector CaMV 35S::GHFLS (vector name pBinGFP4) was constructed, and Arabidopsis thaliana was infected by dipping flowers. The positive plants were identified by kanamycin sulfate screening and PCR detection. By way of selfing and screening, homozygous T2 positive clones were obtained. The root lengths of different strains of Arabidopsis thaliana overexpressing GHFLS and that of wild-type were compared, and it was found that the root length of overexpressed Arabidopsis thaliana was significantly shortened (FIG. 5). The results further prove that the GHFLS gene plays an important role in cell elongation mechanism.

It can be seen from the above results that the gene GHFLS has important research value in improving cotton quality traits and cultivating new varieties of high-quality cotton fibers. On the one hand, molecular markers can be designed according to the haplotype of the gene GHFLS, so as to effectively identify cotton quality traits, which has a good application value in the research of breeding high-quality fiber cotton varieties. On the other hand, the gene containing high-quality haplotype AA can be transferred into cotton varieties by means of genetic engineering to improve cotton quality, or the SNP locus in haplotype GG can be subjected to site-specific mutagenesis and transformed to a high-quality haplotype to cultivate new cotton varieties of high-quality fibers.

TABLE 1
Identification of haplotypes with high fiber quality
and low fiber quality in population varieties

| Haplotype | Name of variety (line) | | | |
|---|---|---|---|---|
| AA | Guannong No. 1 | Jinyu No. 5 | Han cotton 559 | Arcot-1 |
| High | Si-1 | Xinluzao No. 41 | Han 685 | Deltapine |
| | | | | cotton 16 |
| quality | Xinluzao No. 10 | Jinxiu cotton | 99B | DP99B |
| fiber | | No. 1 | | |
| | Jinke 707 | Jiang No. 1 | Han 5158 | Zhongzhi cotton |
| | | | | No. 2 |
| | Xinluzhong | Acala SJ-5 | Liao 96-63-70 | D208 |
| | No. 35 | | | |
| | Liao cotton 10 | DELFOS 531C | Ji 151 | Handan 109 |
| | Zhongmiansuo 16 | Su cotton 22 | Chuangzao 2 | 86-1 |
| | Ji cotton 616 | Ji 668 | Guoxin cotton 11 | Jinzi cotton |
| | Xinluzao No. 12 | Er cotton 14 | L0014 | Ji 1516 |
| | Qin 514 | Han 7860 | Deltapine | Yu cotton No. 8 |
| | | | cotton 15 | |
| | Heishan cotton | 601 | Department 7 | Erjing No. 1 |
| | No. 1 | | | |
| | Wan cotton 73-10 | Jin cotton 28 | Original 32 | Zhongmiansuo 79 |
| | Wan cotton 17 | Wan cotton No. 3 | Xinluzao No. 3 | Zhongmiansuo 35 |
| | Zhongmiansuo | Jin cotton 29 | Xinluzao No. 21 | Yu cotton 20 |
| | No. 45 | | | |
| | Xiang cotton 13 | Xinshi H8 | Xiaoxian Daling | Xinluzao No. 59 |
| | Sheng cotton | Xilunzhong | Yu cotton 15 | Zhongmiansuo 17 |
| | No. 1 | No. 40 | | |
| | Xinluzhong | Yinshan No. 6 | Sun Zhong | Zhongmiansuo 30 |
| | No. 13 | | long staple | |
| | Huiyuan 15-1 | Xinzhi cotton | Lu cotton No. 9 | GK99-1 |
| | | No. 5 | | |
| | Xiang cotton | Brazil 001 | Zhongmiansuo | Yu cotton 11 |
| | No. 11 | | No. 9 | |
| | Cooperative | Liao cotton No. 1 | Yu cotton 19 | Shiduan 5 |
| | cottonseed | | | |
| | in Brazil | | | |
| | Zhong 50 | Liao cotton 9 | SGK321 | Chuan 01 |
| | Western Indian | D201 | Zhongmiansuo 49 | Lumianyan 28 |
| | cotton | | | |
| | Brazil wool | 9D208 | Zhongmiansuo 27 | Ao cotton 618 |
| | Ji cotton No. 1 | Xinluzao No. 47 | Xinluzao No. 22 | Zhongmiansuo 25 |
| | Su cotton No. 5 | Xinzhong No. 41 | Guoxin cotton 3 | Xuzhou 142 |
| | Liu cotton No. 2 | 29-64 | Hua 101 | Si cotton No. 3 |
| | 2010 cited | Bazhou 5409 | Si cotton 2B | Chuan 65 |
| | Ji cotton No. 7 | Zhonglvxu No. 1 | LE-6 | Nongxin No. 1 |
| | Shan 401 | Zhengnong cotton | Su cotton No. 2 | Shaanxi 920346 |
| | | No. 4 | | |
| | Chuan cotton 56 | Jin cotton 19 | Ejing 92 | Xinluzao No. 15 |
| | Xinluzao No. 6 | GK164 | Yun 219 | E cotton 17 |
| | HZ06331 | Zhong 915 | M14 thin wool | K7 |
| | Xinluzhong No. 7 | 14JSC3 | Yu cotton No. 5 | Renhe No. 39 |
| | Moyu No. 1 | Xian III9704 | Er cotton 21 | KK 1543 |
| | Eguang cotton | Xiang SC-24 | Zhongmiansuo | 108Φ |
| | | | No. 3 | |
| | Er cotton No. 4 | Shan cotton No. 1 | Yuzao 95-408 | Daling cotton |
| | | | | (Chaoyang) |
| | Lu cottonyan 22 | Huazhong 910102 | Chuan 267 | 611 [symbol] |
| | Xinluzhong No. 9 | Zhongmiansuo | Er cotton 20 | 602 |
| | | No. 42 | | |
| | Chuan cotton 45 | Zhongmiansuo | Su cotton 9 | Wangjiang long- |
| | | No. 64 | | staple cotton |
| | Shizao No. 2 | Zhongmiansuo 41 | cotton 20 | Brazil 004 |
| | PAYMASTER54 | E cotton 16 | Ji cotton 15 | Xinzhong No. 21 |
| | E cotton 10 | Ba cotton No. 3 | Su cotton 15 | Shan cotton No. 6 |
| | Huihe 38 | NC20B | Ji cotton 11 | COKER'S |
| | | | | FOSTER |
| | Er cotton 12 | 217 | E cotton 2 | Xinzhong No. 23 |
| | Erkang cotton 3 | Ji 122 | Zhongmiansuo 44 | Hua cotton No. 5 |
| | Erkang cotton 9 | Zhongmiansuo | Erjing 8891 | Zheng cotton 18 |
| | | No. 58 | | |
| | Lu cotton 10 | Fu cotton No. 6 | Han cotton 802 | Liao 61107 |
| | Xinluzao No. 39 | Lu cottonyan 16 | Lu cotton No. 1 | Kangsanxing |
| | | | | peach |
| | Ji cotton 10 | Zhong 412 | Xinluzao No. 29 | Wan cotton No. 9 |
| | New B1 | Shiyuan 321 | Brazil 007 | Xinluzao No. 58 |
| | Xinluzao No. 40 | Lanker | 207 | Lu cottonyan 32 |
| | Xuzhou 514 | Zhongmiansuo 23 | cotton No. 1 | Su cotton 11 |
| | TM-1 | 52-128 | Brazil 015 | Xinzhong No. 29 |
| | Xinzhong No. 45 | Si cotton No. 1 | Brazil 0101 | 4005 |
| | Xinluzao No. 49 | Zhongmiansuo 19 | Xuzhou Banban | Antiyellowing |
| | | | cotton | |
| | Indian bract leaf | Jin cotton 12 | LE | Xinzhong No. 26 |
| | Ke 178 | JB298 | Zhongmiansuo | Yuan 93 |
| | | | No. 10 | Kang 393 |
| | Xinluzhong No. 1 | Changrong | Brazil 014 | Zhong 27 |
| | | cotton 69 | | |
| | Xinluzao No. 50 | Han cotton 103 | Guo cotton 9 | Zhengzhou long |
| | | | | staple cotton |
| | Su cotton 20 | 6193 | Xinluzao No. 37 | Bao 6722 |
| | Hu cotton 204 | Han 4104 | Yu cotton No. 1 | K2 |
| | Chuan D9809 | Xinzhong No. 28 | Jing cotton | SGK958 |
| | Xinluzao No. 60 | Zhong C3787 | Dunhuang 77-116 | Xinzhong No. 30 |
| | Xinluzao No. 51 | Aizi cotton 927 | Zhongmiansuo 24 | Su 7235 |
| | Lu cottonyan 29 | Xinluzao No. 24 | Zhongmiansuo 43 | Lu cottonyan 21 |
| | Xuzhou 58 | Zhong 35 | Xuzhou Datao | JCG53 |
| | Shi cotton No. 1 | Zhongmiansuo | DP2824-092 | Yinrui 361 |
| | | No. 50 | | |
| | Shikang 126 | Liao cotton 6611 | 97-4-2 | Department 8-1 |
| | Xinluzao No. 13 | Cotton -2 | Acala SJ-1 | Ji cotton 958 |
| | Xuzhou 209 | Medium C378 | Brazil 011 | Xinzhong No. 47 |
| GG | Xinzhi No. 5 | Xinluzhong No. 3 | 50-15 | Xinzhong No. 19 |
| Low fiber | Xinluzao No. 9 | Sha 2 | Jin cotton No. 6 | TRICE |
| quality | 27-15K | 666 | Xiang cotton 16 | Liao 18 |
| | Xinluzao No. 11 | 97-3-24-1-1 | Su cotton 12 | Zhong 36 |
| | Zhong 30 | Siyang 331 | Line 5 introduced | 46-13 |
| | | | individual plant | |
| | Xinluzao No. 72 | Yanzao No. 2 | Chaoyang cotton | Xinluzao No. 16 |
| | | | No. 2 | |
| | Jin cotton No. 11 | Xinluzao No. 54 | Xinluzao No. 53 | Yinshan No. 8 |
| | Xiang cotton | Xinluzao No. 48 | Liao cotton 17 | Chuan 737-1 |
| | No. 10 | | | |
| | 62-17 | Su cotton No. 3 | Arcot 402bne | Handan 173 |
| | Xinzhong No. 12 | 29-1 | SW1 | 18-3 |
| | Xinluzao 62 | Xinzhong No. 14 | X933 | 609 |
| | Xinzhong No. 11 | Yuzao 73 | 14-21 | Jin cotton No. 2 |
| | Xinluzao No. 5 | Xinluzao No. 61 | Xinluzao No. 46 | Ken 0074 |
| | Xinluzao No. 2 | Xinluzao No. 42 | Liao No. 19 | C1470 |
| | Ji cotton 27 | Liao cotton 19 | Jinzhong 119 | 66-241 |
| | Xinzhong No. 10 | Xinluzao No. 35 | DB3 | Xinluzao No. 18 |
| | Er cotton 6 | Xinluzao No. 7 | Chuan 338 | 9456D |
| | K1 | 98-17 | Beiche No. 1 | 71-7 |
| | Xinluzao No. 32 | Xinluzao No. 8 | Zhong 295-7 | Salt No. 1 |
| | Xinluzhong No. 6 | Xinzhong No. 34 | 115-23 | Jin cotton 18 |
| | Xinluzao No. 26 | Liao 229 | Yinshan No. 7 | Xinzhong No. 20 |
| | Xinluzao No. 38 | Si cotton No. 4 | 98-1 | 9884 |
| | Xinzhong No. 48 | 822 variant strain | Line 7-1 | Xinluzao No. 17 |
| | Xinluzao No. 1 | GK24 | ShiZao3 | Zhong 870203 |
| | Ken 1042 | 9456D-1 | B99621 | Xinzhong No. 32 |
| | Xinluzao No. 33 | Xiang 4108 | Xinluzao No. 28 | Zhong cotton 36 |
| | Yu cotton 18 | Yu cotton 112 | Xinluzao No. 20 | Chaoyang cotton |
| | | | | No. 1 |
| | Y- bud yellow | Jin 10 | Shu cotton No. 1 | Shuihu |
| | | | | cotton 72-8 |
| | 97-3-12-1 | Xinluzao No. 25 | Liao cotton 18 | 2-67 |
| | Jing cotton No. 1 | MA-6 | | |

Sequence Listing

SEQ ID NO. 1: Cotton B3 Transcription Factor Family Gene GHFLS cDNA Sequence

| | |
|---|---|
| atggatcgaa gggtgaagaa ggaagctgaa gagataccgc aaagaacgat gtcatttgct | 60 |
| ggtcggagac ttaaatctgc tggtgaagaa gacttcatcc tcgctctctc aactcacact | 120 |
| cctaagctca acccttcttc ttcggaaaag aaggaaatta gtaagaaggc taatgcgtta | 180 |
| actgagagaa aacagaagcg aaagaagtgc caatccgaga ccataattaa acctgcagtg | 240 |
| tcagattgtg gggagaagaa aataagctct atgaaaaata aggacgtagg tgatggaaga | 300 |
| tgcatagctg aaattaagtc tccagctatg atttgtgcag aggaaattca atcaaaccta | 360 |
| gaacctgaat ttcccagttt tgcaaaatct ttggttagat cacatgtcgg aagctgtttt | 420 |
| tggatggggc ttccggggat gttctgtaaa atacatttac ctaggaaaga tactacaatc | 480 |
| actttggaag acgagagtgg gaaccaattt catgtaaaat actacgctga taaaacggga | 540 |
| ttgagtgcag gttggagaca gttttgtagt gcccataatt tgcttgaggg ggatgttttg | 600 |
| gtcttccagt tagttgagcc aaccaagttc aagatataca taataagggc acatgattta | 660 |
| aatgaattgg atggggctct tggcctccta aatttggatg cttatacaaa acaaagtgat | 720 |
| gcagatgatg cagaaactgg tccaacggtc tctaaaagta caaagaggaa acgtccaaaa | 780 |
| cctcttccac tagcttctgt taggaagaag aacaagaggt ctggcctaca aagattgtct | 840 |
| tgtaacgttg ggcagccggc agagcaatct gaaaatgata gtgaagaagt tggttcagaa | 900 |
| gttttggaag gtttcaagcg aaccgagtct gcaattcaat tcaaagacat aacaagtttc | 960 |
| gagaacatat tggttgatgg cttggttata gatcctgagc tctcggaaga cattcgcagt | 1020 |
| aaatactacc agctatgctg tagtcaaaat gcttttcttc atgaaaatat tatccagggt | 1080 |
| ataagtttta aatttaaagt tggaattatt tccgaaactg tcaatattgc tgatgctata | 1140 |
| agaacttgca agctcacaac ttctcgagat gaatttgata gttgggacag gaccttgaaa | 1200 |
| gcctttgagt tgttgggcat gaatgttggt ttcttacgaa ctcgtcttca ccggcttgta | 1260 |
| aaccttgcat ttgaatcaga aggtgctgct gagacaagga ggtattttga agctaaagca | 1320 |
| gaacgagatc agacagagaa tgagatacga aaccttgaag caaaactcac ggagctgaag | 1380 |
| gatgcaagta aaacctttgg atttgaaatc gagagtttgc aatctaaagc ggaaacaaat | 1440 |
| gaattcaggt ttgagaaaga agttaaggct ccatggtga | 1500 |

SEQ ID No. 2 Cotton B3 Transcription Factor Family Gene GHFLS DNA Sequence

| | |
|---|---|
| atggatcgaa gggtgaagaa ggaagctgaa gagataccgc aaagaacgat gtcatttgct | 60 |
| ggtcggagac ttaaatctgc tggtgaagaa gacttcatcc tcgctctctc aactcacact | 120 |
| cctaagctca acccttcttc ttcggtctct ctctctctct ttcttcttct ttctttactt | 180 |
| cttgctttca gtttgtttag ctggattttg aaacaaaacg atagaaacta aagattctga | 240 |
| aagaaaatat gttagatctc gtacttgttg ctgtagtttt tattttcttc aaaaaagatc | 300 |
| tttagaaggt tcgtactgtc tttgcttgat tgataattat attcattgca ttatatttta | 360 |
| tttttgcagg aaaagaagga aattagtaag aaggctaatg cgttaactga gagaaaacag | 420 |
| aagcgaaaga agtgccaatc cgagaccata attaaacctg taaaaccttt tttcttcctc | 480 |
| ttgttttttt tttgtttaaa tttgttaaat atttttctac ggctattata gaaatattta | 540 |
| tgaccaaatg actcaactgt agtctcaaag ttccaaaatc attgcagaga accaatattt | 600 |
| gaatgattgc tttttttttt cctttctcaa ttcccaagat tgttttgaaa gagtcaaaag | 660 |
| aaaacacata gtagaataat gctaataaat taaacaaaat tggcaactta tgagcaagga | 720 |
| gacagaggta aacttatttt ccatggtcaa actggttgcc gtatggaatg gattagaaca | 780 |
| gagaatttct aatttcaaac ttccagcaag aattgtttgt cttttatctc agattaatta | 840 |
| gcactaacaa ataaatttcc ggacaggcag tgtcagattg tggggagaag aaaataaggt | 900 |
| cagtgaacta tccaaaagaa tggaatctgc gtatgctgtt ttactgcttt tggattaatt | 960 |
| gtctgatgat gtttaccttt tttttaatga agctctatga aaaataagga cgtaggtgat | 1020 |
| ggaagatgca tagctgaaat taagtctcca gctatgattt gtgcagagga aattcaatca | 1080 |
| aacctagaac ctgaatttcc cagttttgca aaatctttgg ttagatcaca tgtcggaagc | 1140 |
| tgtttttgga tggttagctc cgttaaatgc tataattcac cttgtataat tatatttact | 1200 |
| ttttttttga ggtttgtggg atgggtgggg ctaagcctga taacgaaaca gggaatgtgg | 1260 |
| ttggccttaa tcttagtcgc agctgccttg ttggccccat cccctccagc ggcaccctct | 1320 |
| tcctgctcta ccatctccac gagcttaacc ttgcttacag tgatttcaat tggtccccaa | 1380 |
| taggatacca gttttgtcag tttactgtgt tgacccatct aaacatcttt cattaaaaaa | 1440 |
| tttcaggttc aattccatta gtagtctctc acctttctaa actattatcc cttgatctat | 1500 |
| cctatgatga tggtttgatc tttgaagggg atgtcattaa aaatgttgtg ggaaagttga | 1560 |
| cacaactaag acaccttctg ctctcatttg ataggtgtct tagttgtgtc aagtttctca | 1620 |
| tgacattttc caaagagatg cccttcagat ataagtcatt ataggataga ccaagggttg | 1680 |
| atagtttaga aagttgatag attgttaatg gaattttacc tgaaagtttt gattgagaga | 1740 |
| tatttatatg ggtcaacatg gtaaactgac caaactaaga tgctattggg taccaattca | 1800 |
| aataattgta agcaaggtta agcttatgga gatggtggag gaggaagagg gtgctgttag | 1860 |
| tgggcatagg gccaacatag cagttgcaac taagatcaag gcaaatcaca gtacctgttt | 1920 |
| gtgtcacact taaccccacc gcacaaatgt agctctgttt tttgaagttt agcccttgat | 1980 |
| taacttgtaa ataatgaact ttttgaggtg tatttgtgat agtttctttt ttcttttgac | 2040 |
| aggggcttcc ggggatgttc tgtaaaatac atttacctag gaaagatact acaatcactt | 2100 |
| tggaagacga gagtgggaac caatttcatg taaaatacta cgctgataaa acgggattga | 2160 |
| gtgcaggttg gagacagttt tgtagtgccc ataatttgct tgagggggat gttttggtct | 2220 |
| tccagttagt tgagccaacc aagttcaagg tgatgttcaa tagctattct tcttggcttt | 2280 |
| caacttctag agcttgaggt ttatattgct tacgtgcaat atgtgattat atatctgttg | 2340 |
| atagttctgg cttgtcatta gaatttaata ttaagaggac agttccaggt gtgccatatt | 2400 |
| tccccatgtc ccaacattgg atgggcatcc actatgagtg aggggtctga tctgggatct | 2460 |
| gatcgtccaa cctaatatat gatcttctat aacagtattt gttataacat tattagtgat | 2520 |
| taaaaaaaaa gctggtaaaa tacatgtttg gatggttttt ggaaggacaa attggaaaac | 2580 |
| gcaatggaag aaaaatatgg atagagaaac aagggtgcag atgctatcta gttcaggatt | 2640 |
| gtactccttg ccctgtacgt ttcatgttgg cttttgtaga aagtttagca taggagcttc | 2700 |
| ataggtcata tatatacagc ccttaatagc atggatcaat gtctactgaa ttttgtttga | 2760 |
| tctccatctc ttgataatta ataaaggttt aacatgatca tgcatgtatt aagcctatga | 2820 |
| agctcaagtg caaatgccct atgttgttaa agttgaggaa agataggttc tacttttatg | 2880 |
| ctgctgtctt gtataactat atttttgttt caaaaaaggt taaagggtaa aaaaaaaatc | 2940 |
| cttaataaag aaaatcagaa attgattaag tccttatgaa aaaagtaatg cttaattgag | 3000 |
| tcctcggtga ctcaagaaaa ttaattagcc ccttccatta atagaaacct tcgatgatcg | 3060 |
| atttgatcac ggtcattgat gtggtagatg aaagccatga gaatttgaca tgtggctttc | 3120 |
| catatcagca aaaattaaaa aaaattaatt aaaaatttct aaaatatatt taaaaattaa | 3180 |
| ttaaaaatac acatccataa tgaatttaga gatatggacg tcctagttta tttgagttta | 3240 |
| gacacttatt tctttaggtt cattttggct gaacacaaga ctgtaaaaaa tgataaaaca | 3300 |
| ataggtgatg aattgcacca gtcgtagata aaacaatagg tgatcatgta tatctgccga | 3360 |
| aatatggtga aaaaacttac cctatatata cagacagaaa tatgatggta aaactttttc | 3420 |
| cctatttgaa ctctaattta cgaatatatt tcttttatca tgattgtgat ataataaata | 3480 |
| ttaggctaat tttctaccat gttttagcaa atatctatat tatcacttat tgttttactc | 3540 |
| tacagctatt gaagccatat tcatcaccta catatgtgta tatatataac tttttgaatt | 3600 |
| atatattaat tatttatatt atttatagtt ttatgtttgt ccagaacgaa cttggacaaa | 3660 |
| tgtgtccata ttcagatgaa cacggatgta catttgttaa attcattata gatgtgtatt | 3720 |
| tttattaatg agttagaata tttttatatt tctttgaagt ttttattgac attgcatgct | 3780 |
| gcatatcatc ttctcaatgc ttcggtctgc cacattgact gttactggtc aacaacggtt | 3840 |
| ttcgttaacg gaagcggcca attggttttc gtcattgagg aataattggg tgcatttttt | 3900 |
| tataagggct taattgattt tctttttctt tttttttatt aagggtttct tttacctttc | 3960 |
| aaccttttaa aaattaattt gtccgtacaa ctcaaggatt gttcacctta aggtcactgt | 4020 |
| gacgttatga aatgttgtta atttctcaag gtagtggccg ctttagtctt gttacactat | 4080 |
| atataaccaa gtatattaca tacctattgc atttgtattg taaaaataga tgattcacga | 4140 |
| atttttgctg cccccaaatc cacctgcaat tagaaacaat gctagtgaaa atcttgttca | 4200 |
| gcaaaatagt tttggactcc caagaagttg gttgagtaat tttactccta aagactacct | 4260 |
| ttactgttgt gatttttaac ttttatcgtt gatggtcttg aagtgatgag gtctcccttt | 4320 |
| ctcactaaga tctcttttag tgatttacca agcccggaag gtattgaatt ctgggacaac | 4380 |
| atttagataa gaaaggatct tcttaatagt tctaattatt tctggtattt ccatagctaa | 4440 |
| acttgctaaa gaatactggt tctgatcagg tcggattttg cccctcacta gtttgtaaga | 4500 |
| gctgataaac catcagatgc acaaaagctc actgattgtt gcaccaactg aagattttat | 4560 |
| atattttaaa tccaagttaa catagttagt gtttggtaaa atgttgtctg aaacacggtg | 4620 |
| ggcttatgtt taactgacaa ccatataact tttcaaatgc atggtgaggt ttgttagcag | 4680 |
| agatgttaag aaccttaatt tttattatga aaccttattc tttgagatgt agttggggcg | 4740 |
| aatgtatgaa acctgaatgt taaagattct gaaaatgtat ttgccttatt atgaggtcac | 4800 |
| acattgaagg gctaagtagg tgaaaaacaa tgtcaggggt tttagatatc taactatacg | 4860 |
| ggatatactg ttttattaag attggtctgt agtacttgag caagccgata tatcactctc | 4920 |
| caaagacttg catatgttgt tttttaagtt tgcttgattt gtaggaggaa tggagagaga | 4980 |
| caatatactt agttccagct aactacgcca gagctttaag ggtgcacttg attgagtgga | 5040 |
| aaagtggaag gagggaatgt caaggtggaa agaaaataaa ttttgaatgt gtttggtggg | 5100 |
| aaagaaaagt gagaggaaag aaaacaaaag agatgactat tttccacctt aatgcatcaa | 5160 |
| aacaaatcat tccaaattgg aatgataaga ggagagaaaa tgagaggtga atttatgcta | 5220 |
| gttaaaattt atgcattttt ctaaggtttc attttctttc tcttattttt atactctacc | 5280 |
| aagcgatgaa tggaaagaaa atttcttttt ctctcaaatt ttccattcta ttccttccta | 5340 |
| ccaagcacat cagtggaaag aaaaattata tttttcatct ttttattttt ctactcgtac | 5400 |
| aattttctat ccctccaatt tttttttctc tctttcaagt aaagcctaga agtttccata | 5460 |
| agaactacat gggtaataga gaactagtgc agagttcata ttatgtttat tccaaatcat | 5520 |
| tatatcagca aaacaaaatg actgcttagc aatgtttcta acctggcccc atcagtatat | 5580 |
| cgtagaacct aagactgcat taatataaga ggatgcaagg aattaggttt cctcctctat | 5640 |
| ttgaagggag attatctttt atttgtttta aatgcatata tttttgtgaa agtacagtta | 5700 |
| tttacattag taattactct ctatctaacg tgtatcatct tatttttgta gatatacata | 5760 |
| ataagggcac atgatttaaa tgaattggat ggggctcttg gcctcctaaa tttggatgct | 5820 |
| tatacaaaac aaagtgatgc aggcaagttg tttggtgtct ttgttaggtc tcttaattat | 5880 |
| catgcttgca tgtcagaagt tttgcattat aactgaattt ctggggaaaa aataatagat | 5940 |
| gatgcagaaa ctggtccaac ggtctctaaa agtacaaaga ggaaacgtcc aaaacctctt | 6000 |
| ccactagctt ctgttaggaa gaagaacaag aggtctggcc tacaaagatt gtcttgtaac | 6060 |
| gttgggcagc cggcagagca atctgaaaat gatagtgaag aagttggttc agaagttttg | 6120 |
| gaaggtttca agcgaaccga gtctgcaatt caattcaaag acataacaag tttcgagaac | 6180 |
| atattggttg atggcttggt tatagatcct gagctctcgg aagacattcg cagtaaatac | 6240 |
| taccagctat gctgtagtca aaatgctttt cttcatgaaa atattatcca gggtataagt | 6300 |
| tttaaattta aagttggaat tatttccgaa actgtcaata ttgctgatgc tataagaact | 6360 |
| tgcaagctca caacttctcg agatgaattt gatagttggg acaggacctt gaaagccttt | 6420 |
| gagttgttgg gcatgaatgt tggtttctta cgaactcgtc ttcaccggct tgtaaacctt | 6480 |
| gcatttgaat cagaaggtgc tgctgagaca aggaggtatt ttgaagctaa agcagaacga | 6540 |
| gatcagacag agaatgagat acgaaacctt gaagcaaaac tcacggagct gaaggatgca | 6600 |
| agtaaaacct ttggatttga aatcgagagt ttgcaatcta aagcggaaac aaatgaattc | 6660 |
| aggtttgaga aagaagttaa ggctccatgg tga | 6720 |

Primer F1 for Amplification of Cotton B3 Transcription Factor Family Gene
GHFLS cDNA Sequence
SEQ ID NO. 3

| | |
|---|---|
| atggatcgaagggtgaagaa | 20 |

Primer R1 for Amplification of Cotton B3 Transcription Factor Family Gene
GHFLS cDNA Sequence
SEQ ID No. 4

| | |
|---|---|
| tcaccatggagccttaact | 20 |

Primer F2 for Amplification of Cotton B3 Transcription Factor Family Gene
GHFLS SNP Sequence
SEQ ID No. 5

| | |
|---|---|
| acgaaaccttgaagcaaaactca | 23 |

Primer R2 for Amplification of Cotton B3 Transcription Factor Family Gene
GHFLS SNP Sequence
SEQ ID NO. 6

| | |
|---|---|
| ttggaggaagccaatttctg | 20 |

What is claimed is:

1. A B3 transcription factor gene GHFLS for simultaneously improving length, strength and elongation of cotton fibers, wherein, a genomic sequence of the gene is as shown in SEQ ID NO. 2; the B3 transcription factor gene GHFLS comprises a non-synonymous mutation SNP locus, which is located at 1391 bp of a coding region sequence, wherein a base of the SNP locus changes from A to G, and the corresponding amino acid changes from Lys to Arg.

2. Use of the transcription factor gene GHFLS according to claim 1 in identifying an upland cotton variety with high-quality fibers.

3. The use according to claim 2, comprising detecting the SNP locus, and selecting a cotton with a base A at 1391 bp of the coding region sequence is as a high-quality fiber cotton variety.

4. The use according to claim 3, wherein, a primer for detecting the SNP locus is specifically an upstream primer as shown in SEQ ID NO. 5 and a downstream primer as shown in SEQ ID NO. 6.

5. Use of the transcription factor gene GHFLS according to claim 1 in culturing a new variety with high-quality cotton fibers by genetic engineering.


SEQUENCE LISTING (6 sequences)

SEQ ID NO: 1 - DNA, 1479 bp, Gossypium hirsutum
  source 1..1479: mol_type = genomic DNA
atggatcgaagggtgaagaaggaagctgaagagataccgcaaagaacgatgtcatttgctggtcggagacttaaatctgctggtgaagaagacttcatcctcgctctctcaactcacactcctaagctcaacccttcttcttcggaaaagaaggaaattagtaagaaggctaatgcgttaactgagagaaaacagaagcgaaagaagtgccaatccgagaccataattaaacctgcagtgtcagattgtggggagaagaaaataagctctatgaaaaataaggacgtaggtgatggaagatgcatagctgaaattaagtctccagctatgatttgtgcagaggaaattcaatcaaacctagaacctgaatttcccagttttgcaaaatctttggttagatcacatgtcggaagctgtttttggatggggcttccggggatgttctgtaaaatacatttacctaggaaagatactacaatcactttggaagacgagagtgggaaccaatttcatgtaaaatactacgctgataaaacgggattgagtgcaggttggagacagttttgtagtgcccataatttgcttgagggggatgttttggtcttccagttagttgagccaaccaagttcaagatatacataataagggcacatgatttaaatgaattggatggggctcttggcctcctaaatttggatgcttatacaaaacaaagtgatgcagatgatgcagaaactggtccaacggtctctaaaagtacaaagaggaaacgtccaaaacctcttccactagcttctgttaggaagaagaacaagaggtctggcctacaaagattgtcttgtaacgttgggcagccggcagagcaatctgaaaatgatagtgaagaagttggttcagaagttttggaaggtttcaagcgaaccgagtctgcaattcaattcaaagacataacaagtttcgagaacatattggttgatggcttggttatagatcctgagctctcggaagacattcgcagtaaatactaccagctatgctgtagtcaaaatgcttttcttcatgaaaatattatccagggtataagttttaaatttaaagttggaattatttccgaaactgtcaatattgctgatgctataagaacttgcaagctcacaacttctcgagatgaatttgatagttgggacaggaccttgaaagcctttgagttgttgggcatgaatgttggtttcttacgaactcgtcttcaccggcttgtaaaccttgcatttgaatcagaaggtgctgctgagacaaggaggtattttgaagctaaagcagaacgagatcagacagagaatgagatacgaaaccttgaagcaaaactcacggagctgaaggatgcaagtaaaacctttggatttgaaatcgagagtttgcaatctaaagcggaaacaaatgaattcaggtttgagaaagaagttaaggctccatggtga

SEQ ID NO: 2 - DNA, 6693 bp, Gossypium hirsutum
  source 1..6693: mol_type = genomic DNA
atggatcgaagggtgaagaaggaagctgaagagataccgcaaagaacgatgtcatttgctggtcggagacttaaatctgctggtgaagaagacttcatcctcgctctctcaactcacactcctaagctcaacccttcttcttcggtctctctctctctctttcttcttctttctttacttcttgctttcagtttgtttagctggattttgaaacaaaacgatagaaactaaagattctgaaagaaaatatgttagatctcgtacttgttgctgtagtttttattttcttcaaaaaagatctttagaaggttcgtactgtctttgcttgattgataattatattcattgcattatattttatttttgcaggaaaagaaggaaattagtaagaaggctaatgcgttaactgagagaaaacagaagcgaaagaagtgccaatccgagaccataattaaacctgtaaaaccttttttcttcctcttgttttttttttgtttaaatttgttaaatatttttctacggctattatagaaatatttatgaccaaatgactcaactgtagtctcaaagttccaaaatcattgcagagaaccaatatttgaatgattgcttttttttttcctttctcaattcccaagattgttttgaaagagtcaaaagaaaacacatagtagaataatgctaataaattaaacaaaattggcaacttatgagcaaggagacagaggtaaacttattttccatggtcaaactggttgccgtatggaatggattagaacagagaatttctaatttcaaacttccagcaagaattgtttgtcttttatctcagattaattagcactaacaaataaatttccggacaggcagtgtcagattgtggggagaagaaaataaggtcagtgaactatccaaaagaatggaatctgcgtatgctgttttactgcttttggattaattgtctgatgatgtttaccttttttttaatgaagctctatgaaaaataaggacgtaggtgatggaagatgcatagctgaaattaagtctccagctatgatttgtgcagaggaaattcaatcaaacctagaacctgaatttcccagttttgcaaaatctttggttagatcacatgtcggaagctgtttttggatggttagctccgttaaatgctataattcaccttgtataattatatttactttttttttgaggtttgtgggatgggtggggctaagcctgataacgaaacagggaatgtggttggccttaatcttagtcgcagctgccttgttggccccatcccctccagcggcaccctcttcctgctctaccatctccacgagcttaaccttgcttacagtgatttcaattggtccccaataggataccagttttgtcagtttactgtgttgacccatctaaacatctttcattaaaaaatttcaggttcaattccattagtagtctctcacctttctaaactattatcccttgatctatcctatgatgatggtttgatctttgaaggggatgtcattaaaaatgttgtgggaaagttgacacaactaagacaccttctgctctcatttgataggtgtcttagttgtgtcaagtttctcatgacattttccaaagagatgcccttcagatataagtcattataggatagaccaagggttgatagtttagaaagttgatagattgttaatggaattttacctgaaagttttgattgagagatatttatatgggtcaacatggtaaactgaccaaactaagatgctattgggtaccaattcaaataattgtaagcaaggttaagcttatggagatggtggaggaggaagagggtgctgttagtgggcatagggccaacatagcagttgcaactaagatcaaggcaaatcacagtacctgtttgtgtcacacttaaccccaccgcacaaatgtagctctgttttttgaagtttagcccttgattaacttgtaaataatgaactttttgaggtgtatttgtgatagtttcttttttcttttgacaggggcttccggggatgttctgtaaaatacatttacctaggaaagatactacaatcactttggaagacgagagtgggaaccaatttcatgtaaaatactacgctgataaaacgggattgagtgcaggttggagacagttttgtagtgcccataatttgcttgagggggatgttttggtcttccagttagttgagccaaccaagttcaaggtgatgttcaatagctattcttcttggctttcaacttctagagcttgaggtttatattgcttacgtgcaatatgtgattatatatctgttgatagttctggcttgtcattagaatttaatattaagaggacagttccaggtgtgccatatttccccatgtcccaacattggatgggcatccactatgagtgaggggtctgatctgggatctgatcgtccaacctaatatatgatcttctataacagtatttgttataacattattagtgattaaaaaaaaagctggtaaaatacatgtttggatggtttttggaaggacaaattggaaaacgcaatggaagaaaaatatggatagagaaacaagggtgcagatgctatctagttcaggattgtactccttgccctgtacgtttcatgttggcttttgtagaaagtttagcataggagcttcataggtcatatatatacagcccttaatagcatggatcaatgtctactgaattttgtttgatctccatctcttgataattaataaaggtttaacatgatcatgcatgtattaagcctatgaagctcaagtgcaaatgccctatgttgttaaagttgaggaaagataggttctacttttatgctgctgtcttgtataactatatttttgtttcaaaaaaggttaaagggtaaaaaaaaaatccttaataaagaaaatcagaaattgattaagtccttatgaaaaaagtaatgcttaattgagtcctcggtgactcaagaaaattaattagccccttccattaatagaaaccttcgatgatcgatttgatcacggtcattgatgtggtagatgaaagccatgagaatttgacatgtggctttccatatcagcaaaaattaaaaaaaattaattaaaaatttctaaaatatatttaaaaattaattaaaaatacacatccataatgaatttagagatatggacgtcctagtttatttgagtttagacacttatttgtttaggttcattttggctgaacacaagactgtaaaaaatgataaaacaataggtgatgaattgcaccagtcgtagataaaacaataggtgatcatgtatatctgccgaaatatggtgaaaaaacttaccctatatatacagacagaaatatgatggtaaaactttttccctatttgaactctaatttacgaatatatttcttttatcatgattgtgatataataaatattaggctaattttctaccatgttttagcaaatatctatattatcacttattgttttactctacagctattgaagccatattcatcacctacatatgtgtatatatataactttttgaattatatattaattatttatattatttatagttttatgtttgtccagaacgaacttggacaaatgtgtccatattcagatgaacacggatgtacatttgttaaattcattatagatgtgtatttttattaatgagttagaatatttttatatttctttgaagtttttattgacattgcatgctgcatatcatcttctcaatgcttcggtctgccacattgactgttactggtcaacaacggttttcgttaacggaagcggccaattggttttcgtcattgaggaataattgggtgcatttttttataagggcttaattgattttctttttctttttttttattaagggtttcttttacctttcaaccttttaaaaattaatttgtccgtacaactcaaggattgttcaccttaaggtcactgtgacgttatgaaatgttgttaatttctcaaggtagtggccgctttagtcttgttacactatatataaccaagtatattacatacctattgcatttgtattgtaaaaatagatgattcacgaatttttgctgcccccaaatccacctgcaattagaaacaatgctagtgaaaatcttgttcagcaaaatagttttggactcccaagaagttggttgagtaattttactcctaaagactacctttactgttgtgatttttaacttttatcgttgatggtcttgaagtgatgaggtctccctttctcactaagatctcttttagtgatttaccaagcccggaaggtattgaattctgggacaacatttagataagaaaggatcttcttaatagttctaattatttctggtatttccatagctaaacttgctaaagaatactggttctgatcaggtgggattttgcccctcactagtttgtaagagctgataaaccatcagatgcacaaaagctcactgattgttgcaccaactgaagattttatatattttaaatccaagttaacatagttagtgtttggtaaaatgttgtctgaaacacggtgggcttatgtttaactgacaaccatataacttttcaaatgcatggtgaggtttgttagcagagatgttaagaaccttaatttttattatgaaaccttattctttgagatgtagttggggcgaatgtatgaaacctgaatgttaaagattctgaaaatgtatttgccttattatgaggtcacacattgaagggctaagtaggtgaaaaacaatgtcaggggttttagatatctaactatacgggatatactgttttattaagattggtctgtagtacttgagcaagccgatatatcactctccaaagacttgcatatgttgttttttaagtttgcttgatttgtaggaggaatggagagagacaatatacttagttccagctaactacgccagagctttaagggtgcacttgattgagtggaaaagtggaaggagggaatgtcaaggtggaaagaaaataaattttgaatgtgtttggtgggaaagaaaagtgagaggaaagaaaacaaaagagatgactattttccaccttaatgcatcaaaacaaatcattccaaattggaatgataagaggagagaaaatgagaggtgaatttatgctagttaaaatttatgcatttttctaaggtttcattttctttctcttatttttatactctaccaagcgatgaatggaaagaaaatttctttttctctcaaattttccattctattccttcctaccaagcacatcagtggaaagaaaaattatatttttcatctttttatttttctactcgtacaattttctatccctccaatttttttttctctctttcaagtaaagcctagaagtttccataagaactacatgggtaatagagaactagtgcagagttcatattatgtttattccaaatcattatatcagcaaaacaaaatgactgcttagcaatgtttctaacctggccccatcagtatatcgtagaacctaagactgcattaatataagaggatgcaaggaattaggtttcctcctctatttgaagggagattatcttttatttgttttaaatgcatatatttttgtgaaagtacagttatttacattagtaattactctctatctaacgtgtatcatcttatttttgtagatatacataataagggcacatgatttaaatgaattggatggggctcttggcctcctaaatttggatgcttatacaaaacaaagtgatgcaggcaagttgtttggtgtctttgttaggtctcttaattatcatgcttgcatgtcagaagttttgcattataactgaatttctggggaaaaaataatagatgatgcagaaactggtccaacggtctctaaaagtacaaagaggaaacgtccaaaacctcttccactagcttctgttaggaagaagaacaagaggtctggcctacaaagattgtcttgtaacgttgggcagccggcagagcaatctgaaaatgatagtgaagaagttggttcagaagttttggaaggtttcaagcgaaccgagtctgcaattcaattcaaagacataacaagtttcgagaacatattggttgatggcttggttatagatcctgagctctcggaagacattcgcagtaaatactaccagctatgctgtagtcaaaatgcttttcttcatgaaaatattatccagggtataagttttaaatttaaagttggaattatttccgaaactgtcaatattgctgatgctataagaacttgcaagctcacaacttctcgagatgaatttgatagttgggacaggaccttgaaagcctttgagttgttgggcatgaatgttggtttcttacgaactcgtcttcaccggcttgtaaaccttgcatttgaatcagaaggtgctgctgagacaaggaggtattttgaagctaaagcagaacgagatcagacagagaatgagatacgaaaccttgaagcaaaactcacggagctgaaggatgcaagtaaaacctttggatttgaaatcgagagtttgcaatctaaagcggaaacaaatgaattcaggtttgagaaagaagttaaggctccatggtga

SEQ ID NO: 3 - DNA, 20 bp, synthetic construct
  source 1..20: mol_type = other DNA
atggatcgaagggtgaagaa

SEQ ID NO: 4 - DNA, 20 bp, synthetic construct
  source 1..20: mol_type = other DNA
tcaccatggagccttaactt

SEQ ID NO: 5 - DNA, 23 bp, synthetic construct
  source 1..23: mol_type = other DNA
acgaaaccttgaagcaaaactca

SEQ ID NO: 6 - DNA, 20 bp, synthetic construct
  source 1..20: mol_type = other DNA
ttggaggaagccaatttctg